(19) United States
(12) Patent Application Publication
(10) Pub. No.: US 2015/0049332 A1
(43) Pub. Date: Feb. 19, 2015

(54) GOLD NANOISLAND ARRAYS

(71) Applicant: The Curators of the University of Missouri, Columbia, MO (US)

(72) Inventors: Xin Sun, Columbia, MO (US); Hao Li, Columbia, MO (US)

(21) Appl. No.: 14/447,220

(22) Filed: Jul. 30, 2014

Related U.S. Application Data
(60) Provisional application No. 61/958,520, filed on Jul. 30, 2013.

(51) Int. Cl. G01N 21/65; G01N 33/24

(52) U.S. Cl. CPC G01N 21/658; G01N 33/24; B82Y 35/00
USPC 356/301; 977/881

(57) ABSTRACT

A substrate for facilitating enhanced SERS analysis, including a semiconducting substrate and a plurality of discrete metal nanostructures disposed on the semiconducting substrate to define an array. Each respective metal nanostructure is between about 10 nm and about 30 nm high and between about 15 nm and about 60 nm in diameter and two adjacent respective metal nanostructures are separated by a gap of between about 20 nm to about 50 nm.

### CROSS-REFERENCE TO RELATED APPLICATIONS

This patent application claims priority to co-pending provisional patent application Ser. No. 61/958,520, filed on Jul. 30, 2013.

### TECHNICAL FIELD

The present invention relates generally to materials science, surface engineering, spectroscopy, and, more particularly, surface enhanced Raman spectroscopy (SERS).

### BACKGROUND

Since the first observation of SERS in 1974, a variety of metal nanostructures have been used to realize the enhancement of Raman signals. Generally, coinage metals such as Au, Ag, and Cu, are among the preferred choices for SERS applications because their dielectric constants satisfy the resonance condition at Raman excitation wavelengths which are typically near to or in the visible region. Among different nanostructures for SERS, nanoscale gaps between adjacent nanoparticles have demonstrated relatively strong enhancement effects and are often referred as “hot spots” for SERS. The enhancement factor, which is defined by the times of enhancement compared to a control, has been considered as one of the primary parameters for characterizing the performance of a SERS substrate. Other important parameters descriptive of SERS substrates include uniformity, repeatability, reproducibility, scalability, shelf life, and cost. In attempting to obtain reliable and scalable SERS substrates, numerous fabrication techniques and approaches have been explored, including various nanogeometries from electron beam lithography, nanopyramids and nanopillars from nanosphere lithography, nanowires decorated with nanoparticles, porous nanoscaffolds, nanoclusters from colloidal aggregation, rough thin films from chemical and physical roughening, nanorods from oblique angle deposition, nanoporous gold from dealloying, and the like. While advances have been made, there is still a need to develop SERS substrates with high enhancement factors, excellent reliability, and good scalability at low cost. The present novel technology addresses this need.

### DESCRIPTION OF DRAWINGS

FIG. 1 is a schematic representation of a method for making SERS substrates according to one embodiment of the present invention.

FIG. 2A is a first photomicrograph of gold nanostructures on the SERS substrates of FIG. 1.

FIG. 2B is a second photomicrograph of gold nanostructures on the SERS substrates of FIG. 1.

FIG. 2C is a third photomicrograph of gold nanostructures on the SERS substrates of FIG. 1.

FIG. 2D is a fourth photomicrograph of gold nanostructures on the SERS substrates of FIG. 1.

FIG. 2E is a fifth photomicrograph of gold nanostructures on the SERS substrates of FIG. 1.

FIG. 2F is a sixth photomicrograph of gold nanostructures on the SERS substrates of FIG. 1.

FIG. 3 is a graphic illustration of the size distribution of nanostructures after single, double, and triple deposition according to the method of FIG. 1.

FIG. 4A graphically illustrates the single SERS surface of FIG. 3.

FIG. 4B graphically illustrates the double SERS surface of FIG. 3.

FIG. 4C graphically illustrates the triple SERS surface of FIG. 3.

FIG. 5A is a first SEM image of gold nanostructures before annealing.

FIG. 5B is a second SEM image of gold nanostructures before annealing.

FIG. 5C is a photomicrograph of the gold nanostructures of FIG. 5A after annealing.

FIG. 5D is a photomicrograph of the gold nanostructures of FIG. 5B after annealing.

FIG. 6 graphically illustrates Raman shift vs. Raman intensity for single, double, and triple deposited SERS surfaces according to FIG. 1.

FIG. 7A graphically illustrates Raman shift vs. Raman intensity for triple processed SERS substrates of FIG. 1 and having nanostructures of varying thicknesses.

FIG. 7B graphically illustrates gold thickness vs. normalized signal intensity for the SERS substrates of FIG. 1.

FIG. 8A is a first SEM image of gold nanoislands according to FIG. 1.

FIG. 8B is a second SEM image of gold nanoislands according to FIG. 1.

FIG. 8C is a third SEM image of gold nanoislands according to FIG. 1.

FIG. 8D is a fourth SEM image of gold nanoislands according to FIG. 1.

FIG. 8E is a fifth SEM image of gold nanoislands according to FIG. 1.

FIG. 9A graphically Raman shift vs. Raman intensity for triple deposited SERS surfaces with 40 nm gold deposition according to FIG. 1.

FIG. 9B graphically analyte concentration vs. Raman intensity for the substrate of FIG. 9A.

FIG. 10 graphically illustrates the distribution of Raman intensity over a SERS substrate according to FIG. 1.

### DETAILED DESCRIPTION

For the purposes of promoting an understanding of the principles of the invention and presenting its currently understood best mode of operation, reference will now be made to the embodiments illustrated in the drawings and specific language will be used to describe the same. It will nevertheless be understood that no limitation of the scope of the invention is thereby intended, with such alterations and further modifications in the illustrated device and such further applications of the principles of the invention as illustrated therein being contemplated as would normally occur to one skilled in the art to which the invention relates.

Optical-based sensing has several major advantages over electronic sensing because optical sensing reveals spectral fingerprints of chemical compounds rapidly and accurately, thus significantly simplifying the detection process and reducing false alarms. One of the most promising optical sensing techniques is surface enhanced Raman spectroscopy (SERS), which employs noble metal nanostructures to dramatically enhance Raman signals. With the aid of metallic nanostructures, such as gold based nanosubstrates, a Raman signal may be enhanced by a factor of 10⁴ to 10⁸ times, or even higher. This enhancement is due to the generation of spatially localized surface plasmon resonance (SPR) “hot spots” where huge local enhancements of electromagnetic field are obtained. The location of “hot spots” on the metallic structures depends on the geometry of the nanostructures, the excitation wavelength, and polarization of the optical fields. SERS can potentially reach the limit of detection down to the low parts-per-billion (ppb) and theoretically to the single molecule level. Thus, SERS has been increasingly used as a signal transduction mechanism in biological and chemical sensing.

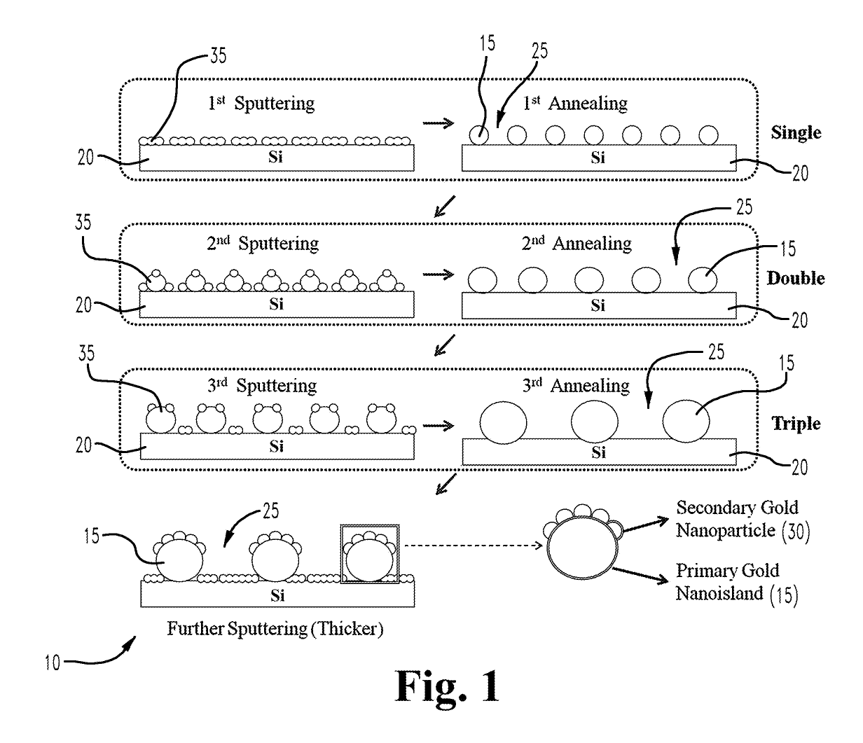

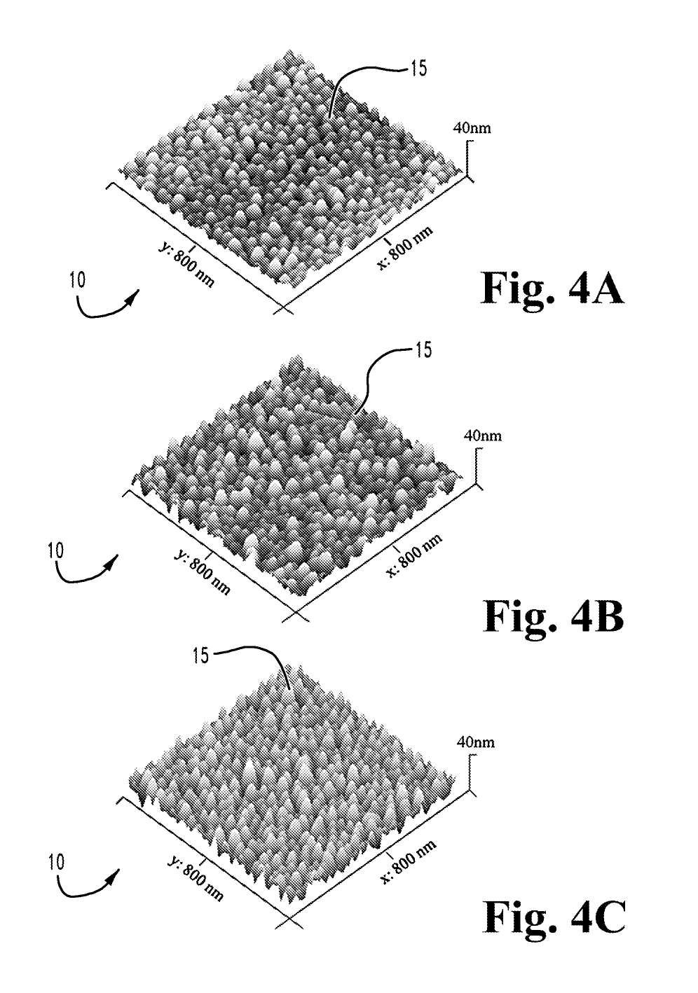

The novel technology, as illustrated in FIGS. 1-10, relates to an improved SERS substrate or surface 10 and method for producing the same. Specifically, the novel technology relates to a simplified substrate fabrication technique utilizing controllable sputtering and heat treatment processes to yield quasi-periodic arrays of gold nanostructures 15 or ‘nano-islands’ on an underlying substrate 20 without having to resort to more complicated and expensive processes such as lithography, templates, or the like.

According to one embodiment of the present novel technology, the SERS surface 10 may include a base substrate 20 and a plurality of metallic nanostructures 15 deposited upon the base substrate 20. The substrate 20 is typically a semiconducting material, such as silicon. The nanostructures 15 are typically formed from gold, although other metals, such as silver, platinum, palladium, copper, nickel, titanium, chromium, or the like, and combinations thereof, may also be selected. The nanostructures 15 are typically sputtered onto the substrate, and are typically more or less uniformly sized and distributed. The plurality of more or less evenly distributed and sized nanostructures 15 likewise defines a plurality of gaps 25 therebetween. The respective gaps 25 may be free of nanostructure metal, or may simply contain less of it than do the adjacent nanostructures 15.

The nanostructures 15 are typically between about 10 nm and about 30 nm high and between about 15 nm and about 60 nm in diameter, more typically between about 20 nm and about 30 nm high and between about 30 nm and about 60 nm across. The nanostructures 15 define separation gaps 25 therebetween of between about 20 nm to about 50 nm. The gaps 25 may be totally devoid of the nanostructure metal, or may contain small thicknesses (typically less than about 5-10 nm) of the same, wherein those small thicknesses are substantially smaller than the nanostructure heights.

Typically, the nanostructures 15 are substantially strain-free, such as from having been annealed. The nanostructures 15 are likewise typically circular or quasi-circular in shape (as viewed from above), and typically have rounded features, although in some cases the nanostructures may have sharp features or combinations of sharp and rounder features. The nanostructures 15 may include additional roughening or protrusions 30 extending therefrom. The protrusions 30 are typically rounded (but may alternately be sharp) and are typically added by sputtering additional metal material onto previously formed nanostructures 15. Protrusions 30 are typically about 5 nm across.

Improved SERS surfaces 10 are typically produced by sputtering or otherwise introducing 40 gold or a like metal 35 onto a semiconducting substrate 20 to yield a plurality of generally evenly or homogeneously distributed nanostructures 15 defining a plurality of (typically generally evenly distributed) gaps 25 therebetween. The nanostructures 15 are then annealed 45. Typically, the sputtering/annealing processes 40/45 are repeated at least once, more typically at least twice, to yield a plurality of annealed nanoislands 15 on the substrate 20. While the last step is typically an annealing step 45, it may likewise be a sputtering step 40. Further, while each subsequent sputtering step 40 typically introduces more of the same metal 35 as did the previous step 40, in some embodiments subsequent sputtering 40 introduces different metal 35.

### EXAMPLE 1

A silicon (100) wafer 20 was rinsed by acetone, methanol, and de-ionized water sequentially, and then dried by nitrogen gas flow. An ultra-thin non-contiguous gold layer defining a plurality of nanostructures 15, and with a 5 nm nominal thickness, was sputtered 40 on the wafer 20. The as-coated silicon wafer 20 was annealed 45 in a quartz tube furnace at 200° C. for 2 h, during which 100 standard cubic centimeters per minute (sccm) mixed forming gas (95% argon and 5% hydrogen) was used as the protective agent. After the first annealing 45, a plurality of gold nanoisland structures 15 had been formed. The 5 nm gold sputtering deposition 40 was repeated, as was a post-deposition annealing 45 of the gold nanoisland structures 15 either one or two times more, yielding three samples respectively having pluralities of gold nanoislands 15 with single, double, and triple sputtering deposition 40 and post-deposition annealing 45 processes, respectively. The triple processed gold nanoislands 15 were coated with a final layer of gold thin film (10 nm, 20 nm, 30 nm, 40 nm, 50 nm, and 60 nm nominal thicknesses) for the SERS measurements. For convenience, the gold nanoislands after each annealing 45 are referred to as primary gold nanoislands 15′ and the final layer of gold deposition is referred to as secondary gold nanoparticles 15″. The secondary gold nanoparticles 15″ are smaller than the primary gold nanoislands 15′. In addition, for comparison, a single process of gold sputtering deposition 40 and post-deposition annealing 45 was also applied on samples with initial nominal thickness of 10 nm and 15 nm. The planar morphology of gold nanoislands 15 at different stages was characterized with field emission SEM, and the heights of gold nanoislands 15 were measured with tapping mode atomic force microscopy. The size distribution analysis of gold nanoislands 15 was carried out by measuring and counting nanoislands across 1.2 μm by 1.2 μm SEM images.

Raman spectra were collected using a 20× objective with 10-second exposure time and ˜50 mW laser output. Malachite green (MG) and 1,2-benzenedithiol (1,2-BDT) were used as analytes. Analyte solutions of MG at various concentrations were prepared in an acetonitrile and water mixture (1:1 in volume). For each measurement, 0.2 μl MG analyte solution was dropped onto a SERS substrate 10 and dried in air. Also, a self-assembled monolayer (SAM) of 1,2-BDT was formed on a SERS substrate 10 in order to study its uniformity. Given the inner diameter (5.08 cm) of the tube used for annealing, the dimension of this SERS substrate was confined at 5 cm by 5 cm.

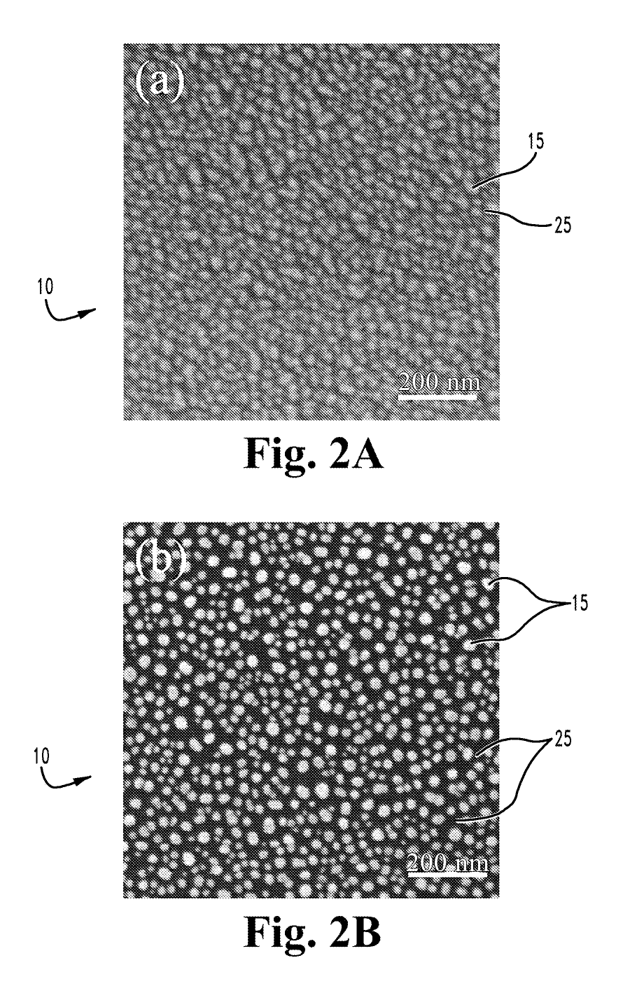

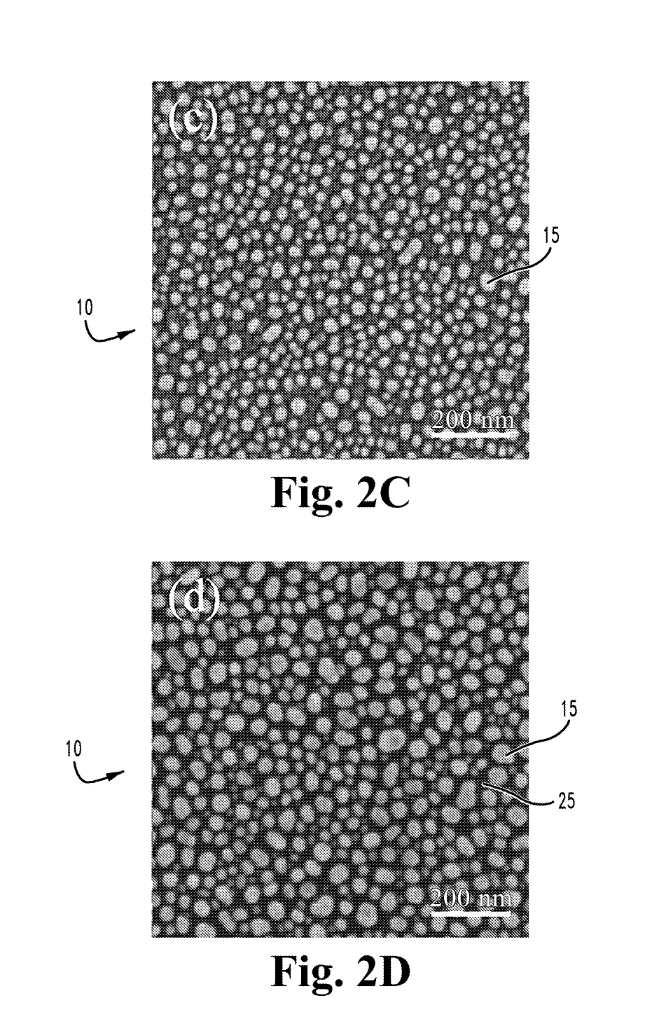

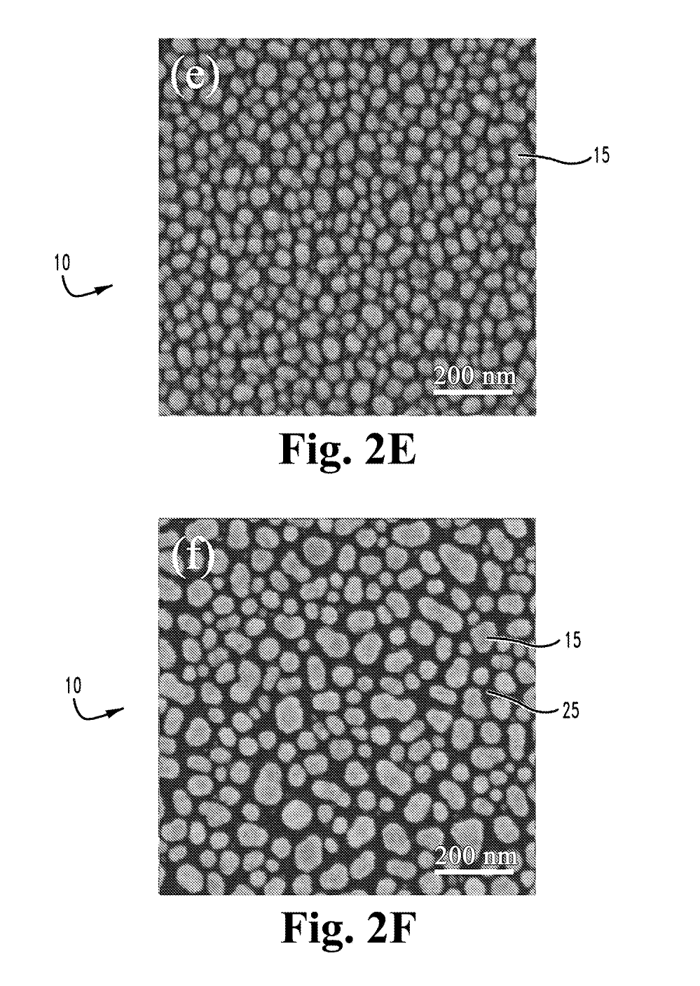

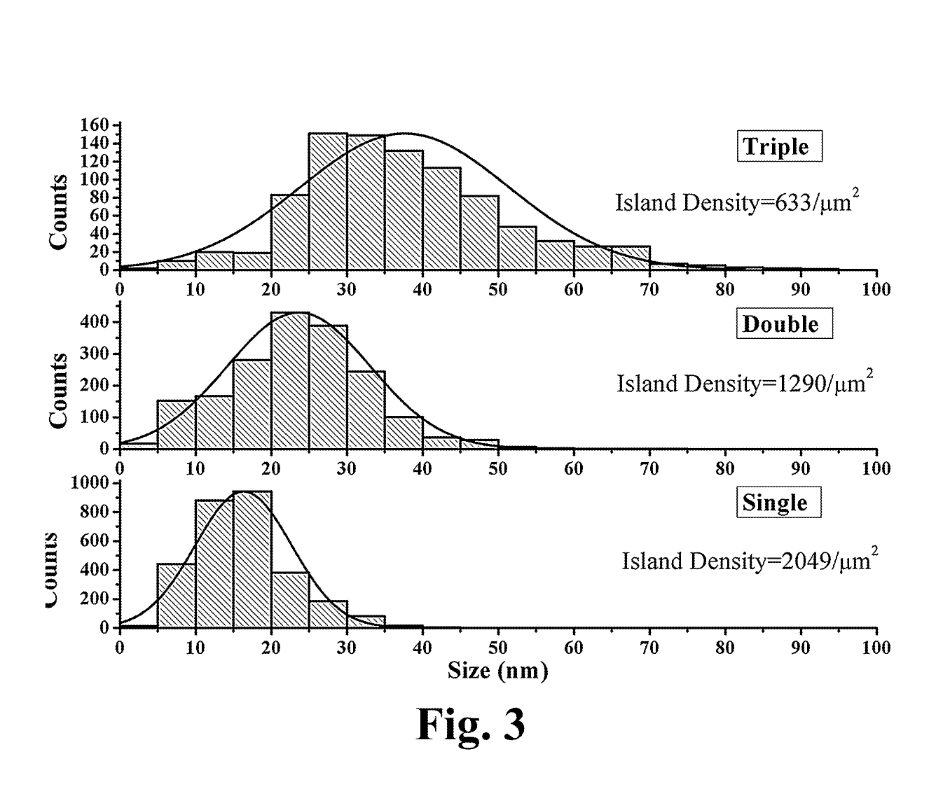

SEM characterization showed nearly round nanoislands 15 (from top view) formed with an average diameter of ˜16 nm after a single sputtering deposition 40 (5 nm nominal thickness) and post-deposition annealing process 45 (FIG. 2A corresponding to “1^{st} sputtering 40” and FIG. 2B to “1^{st} annealing 45” in FIG. 1). After this process was repeated one time (FIG. 2C corresponding to “2^{nd} sputtering 40” and FIG. 2D to “2^{nd} annealing 45 ” in FIG. 1) and two times (FIG. 2E corresponding to “3^{rd} sputtering 40” and FIG. 2F to “3^{rd} annealing 45” in FIG. 1), the average diameter of nanoislands 15 grew to ˜24 nm and ˜38 nm, respectively. In other words, the size of nanoisland 15 was controlled by additional process of deposition 40 and annealing 45, as also evidenced by the histogram in FIG. 3. The total number of nanoislands 15 declined (values shown in FIG. 3) as the nanoislands 15 grew bigger, indicative of merging among adjacent nanoislands 15 during the subsequent annealing process 45. This merging effect, along with the new sputtered gold, contributed to the growth of nanoislands 15. In addition, the vertical height of nanoislands 15 increased with repeated deposition 40 and annealing 45 processes. Specifically, the average heights of single, double, and triple processed nanoislands 15 were ˜11 nm, ˜15 nm, and ˜24 nm, respectively. The heights of the nanoislands 15 were smaller than their corresponding planar diameters in all three samples.

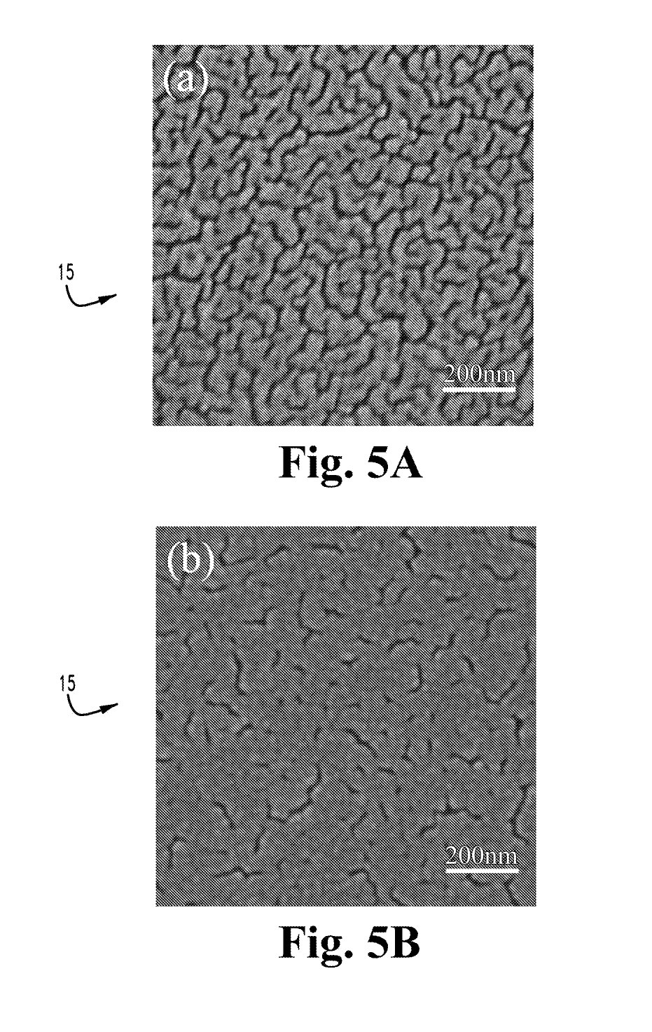

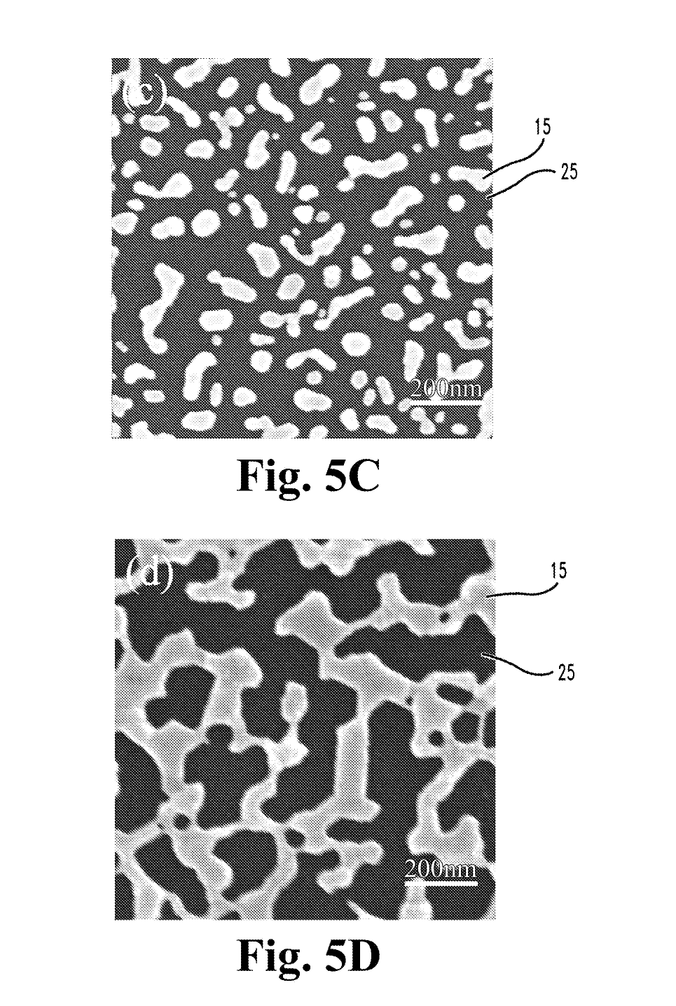

By contrast, a single process of sputtering deposition 40 and post-deposition annealing 45 was also applied on samples with initial nominal thicknesses of 10 nm (equivalent to 2×5 nm) and 15 nm (equivalent to 3×5 nm). The planar morphology of these two thin films before annealing is shown in FIGS. 5A and 5B, respectively. After annealing 45, FIGS. 5C and 5D depict that the resulting structures 15 were larger, non-uniform, and irregular in shape. Thus, repeated deposition 40 and annealing 45 processes yield larger nanoislands 15 with increased uniformity and shape consistency.

While sputtered gold is composed of tiny nanoparticles with diameters of a few nanometers, gold sputtered onto substrates evolves to completely covered thin film state through a series of stages, namely isolated islands, percolation, holes filling, and finally thin film. When induced by annealing, the sputtered gold transforms into larger islands. This process is referred as to solid-state dewetting. The reduction of surface free energy (through the reduction of surface area) and the difference in thermal expansion coefficient between gold and the silicon substrate are the driving forces for the morphology change. Specifically, the inhomogeneous stress distribution at the gold-silicon interface promotes the migration of gold atoms to more relaxed regions. In addition, elevated temperatures enhance the mobility of gold atoms and facilitate small gold nanoparticles coalescing into larger islands to reduce the surface free energy. In general, the morphology of the resulting nanoislands 15 is primarily influenced by two factors, the initial film thickness or the initial stage of the sputtered gold (isolated islands, percolation, or holes filling), and the annealing environment, including temperature, atmosphere, and time.

In the above Example, 5 nm gold deposition 40 produced isolated nanoislands 15 (FIG. 2A), 10 nm gold deposition 40 produced percolation (FIG. 5A), and 15 nm gold deposition 40 was at the stage of holes filling (FIG. 5B). Since the annealing environment was unchanged, the initial film thickness accounted for the morphological variation of gold nanoislands 15 (FIGS. 2B, 5C, and 5D).

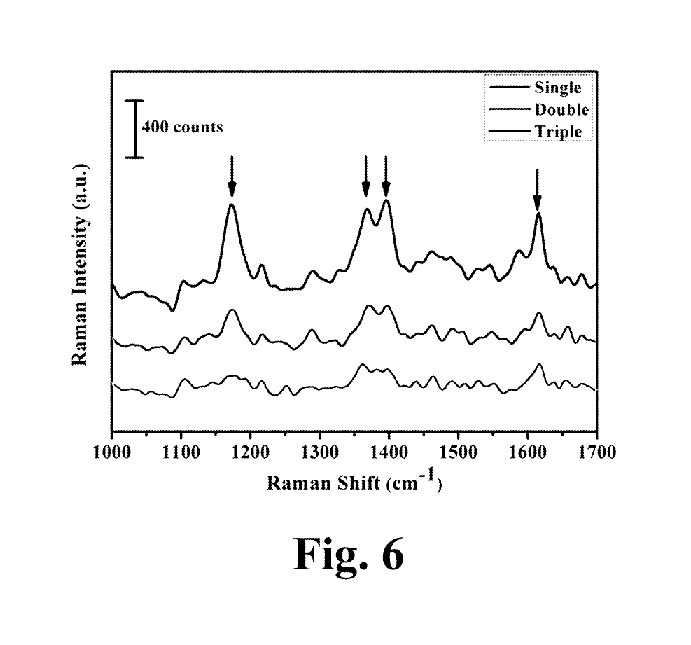

The size and spacing of gold nanoparticles strongly affect localized surface plasmon resonance (LSPR) which constitutes the foundation of electromagnetic enhancement of SERS. With interparticle coupling effect being neglected, an isolated spherical gold nanoparticle 15 yields the maximum enhancement when particle diameter is ˜60 nm. Before reaching the maximum, the enhancement increased with increasing particle size. Regarding to spacing, with the particle diameter being fixed, the interparticle coupling effect starts to take place when the particle center to center distance is less than twice the particle diameter, and the effect then rises dramatically with decreasing interparticle distance. In other words, once interparticle coupling takes effect, SERS enhancement increases sharply with an increasing ratio of particle diameter to interparticle distance. Although a smaller interparticle distance generally leads to higher enhancement, the chances for molecules to locate in such a “hot spot” zone may also decrease. As a result, there is an optimal interparticle distance, not necessarily the smallest, that provides the highest enhancement for the substrates 10. FIG. 6 shows the SERS spectra of 2 parts per million (ppm) MG on these single, double, and triple deposition and annealing processed substrates 10. As indicated in the spectra, the triple processed substrate 10 exhibited the highest enhancement. As the process was repeated, the nanoisland diameter increased towards 60 nm, the preferred size for maximum SERS performance when not considering particle interactions. Secondly, the nanoisland diameter to inter-island distance ratio increased as well (FIGS. 2B, 2D, and 2F). Both these two factors indicated the triple processed substrate 10 as the best one for SERS purposes.

### EXAMPLE 2

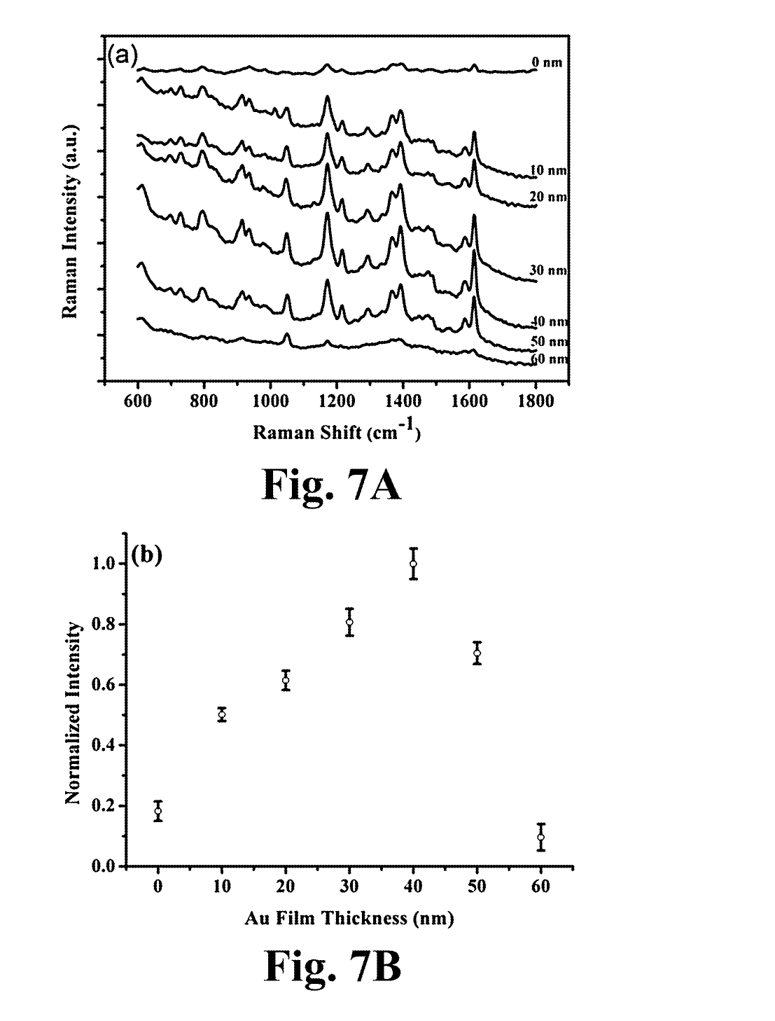

A series of gold thin films (10 nm, 20 nm, 30 nm, 40 nm, 50 nm, and 60 nm nominal thicknesses) were deposited over the primary gold nanoisland 15 arrays, with the thin films actually made of nanoparticles of a few nanometers. The nanoisland 15 arrays were produced as described above with the triple deposition 40 and annealing 45 process, which generated the best performance prior to the final layer of gold thin film deposition. In order to investigate the influence of the thin film thickness on SERS performance, these substrates 10 were tested by measuring 200 parts per billion (ppb) MG with all other experimental conditions held constant. FIG. 7A shows that the Raman signal initially increased as the film became thicker and then started to decrease when the film was thicker than about 40 nm. For confirmation, a quantitative analysis was performed by normalizing the Raman intensity (peak height) at band of 1173 cm⁻¹. In details, the peak height of the 40 nm sample was set as Iₘₐₓ and the normalized Raman intensity was defined by dividing all peak heights by Iₘₐₓ. The relationship between film thickness and normalized Raman intensity (I/Iₘₐₓ) is presented in FIG. 7B. The graph further suggests the optimal thickness lies at ˜40 nm. The final layer of gold thin film appears to have exerted two opposite effects on the SERS performance. One of them was to boost the SERS performance with (i) the improved surface roughness at the few nanometer scale through the addition of secondary gold nanoparticles and (ii) the decrease of spacing between primary gold nanoislands with relatively thin gold coating, while the competing effect was to degrade the SERS performance with the reduction of roughness at the tens of nanometer scale as the gaps 25 between primary gold nanoislands were closed up with a thicker gold coating.

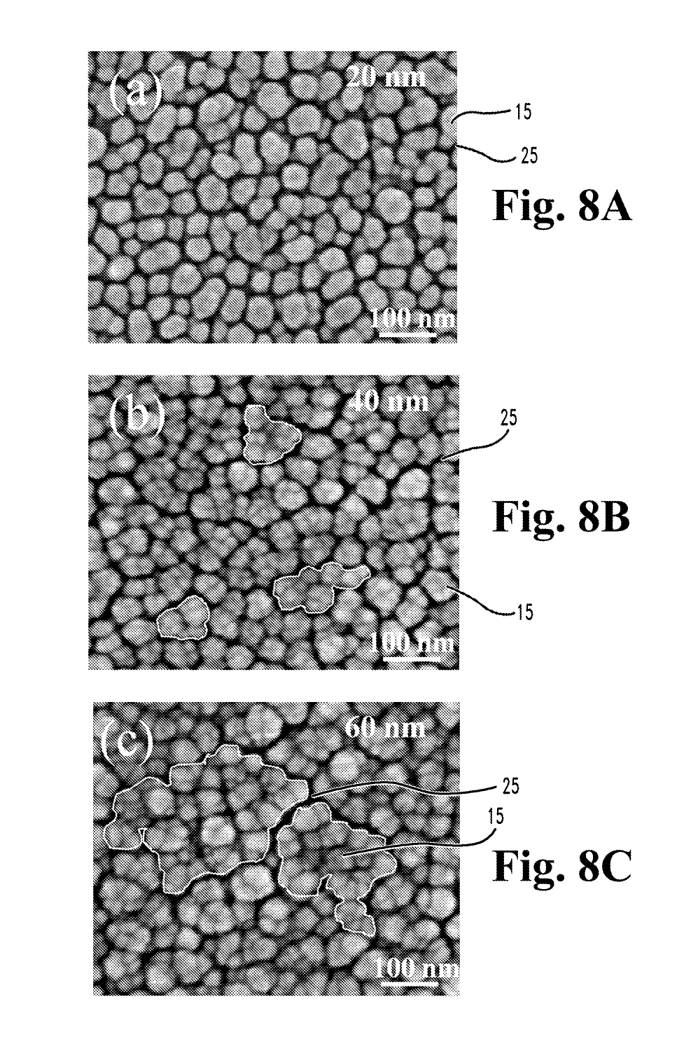

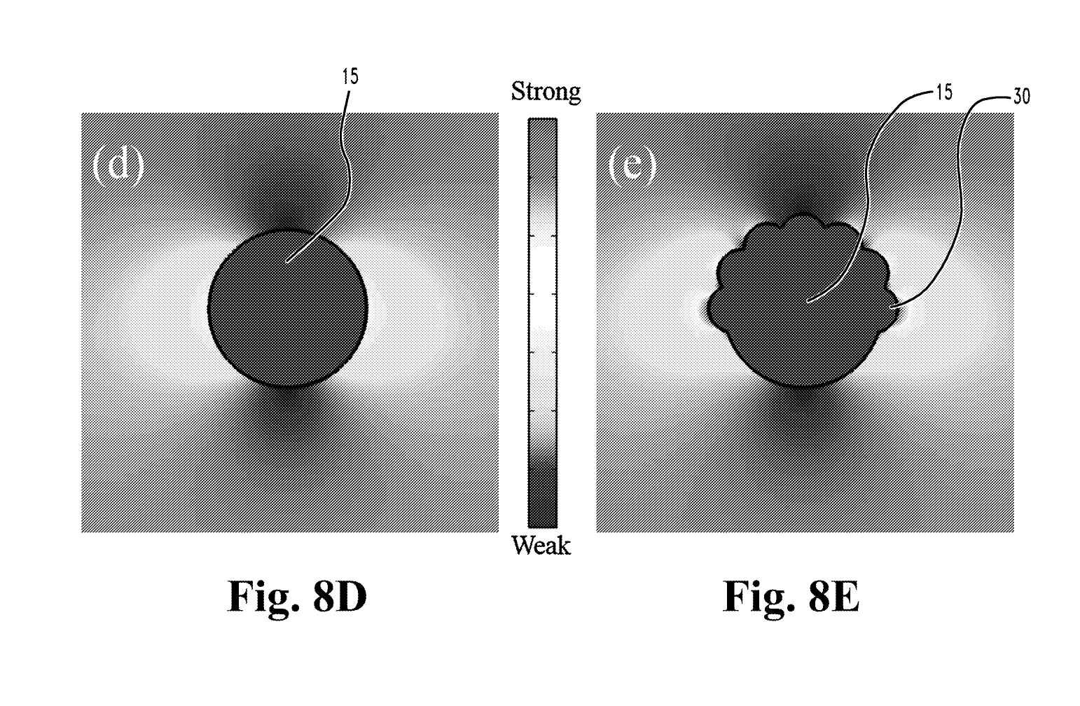

As shown in FIGS. 8A-C, the surfaces of primary nanoislands 15′ remain relatively clean and smooth after 20 nm gold deposition, and then became rougher after 40 and 60 nm gold depositions. In other words, the gold deposition gradually introduced small secondary nanoparticles 30 on top of the primary nanoislands 15′, resulting in the addition of roughness at the few nanometer scale. Finite element method simulation of scattered electric field with a vertical incident plane wave (λ=785 nm) was carried out on two equally sized objects: a smooth sphere and a secondary nanoparticle-coated sphere. FIGS. 8D and 8E demonstrate that the latter with additional roughness induced a more intense scattered electric field close to sphere surface, which suggests secondary nanoparticle-coated nanoislands 15″ produce higher Raman enhancement. The simulation on individual primary nanoislands 15′ favored 40 and 60 nm films for superior SERS performance.

Gaps 25 between adjacent nanoparticles 15 are believed to be the region where the highest enhancement is generated, and are thus often referred as SERS “hot spots”. As shown in FIG. 8A, the primary nanoislands 15 remained separated from each other, and, in turn, many gaps 25 were present after 20 nm gold deposition. In addition, this 20 nm gold film made the gaps narrower, which ultimately improved the SERS performance due to a higher ratio of particle diameter to interparticle distance. When the film thickness reached 40 and then 60 nm, however, the gaps 25 gradually disappeared as they were filled by the gold deposition. As marked in FIGS. 8B and 8C, some initially isolated primary nanoislands 15 merged into bigger nanoislands 15. As a consequence, the density of so called “hot spots” declined due to the loss of gaps 25, and the SERS performance was adversely affected.

Optimally, there should be a balance of the positive and negative effects of the final layer gold deposition 40. In fact, the SERS measurement in FIG. 7 agrees upon the existence of such compromising behavior in that the optimal thickness lies between too thin and too thick coatings.

### EXAMPLE 3

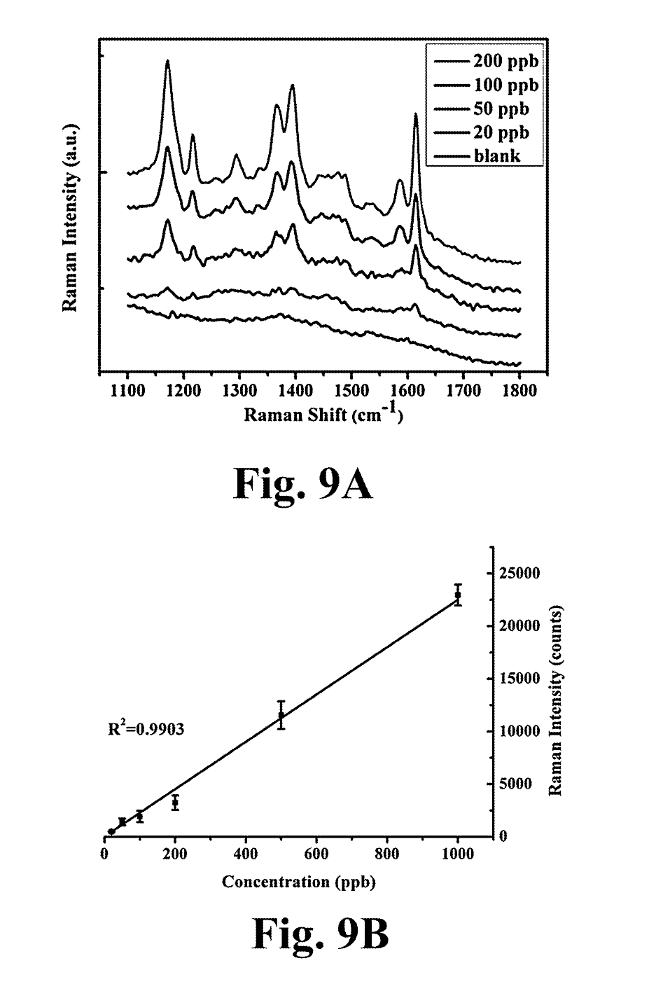

Triple processed gold nanoisland 15 arrays with 40 nm gold deposition were used for detection limit analysis. FIG. 9A illustrates stacking spectra of MG analytes, along with a blank scan for reference. As shown, the responses at bands of 1173 cm⁻¹ and 1615 cm⁻¹ were still observable at concentrations as low as 20 ppb. The relationship between Raman intensity (1615 cm⁻¹) and analyte is shown in FIG. 9B. For each concentration, five spectra were collected and the corresponding peak heights were recorded. The linear behavior (R²=0.9903) of this substrate suggests good potential for quantitative SERS analysis.

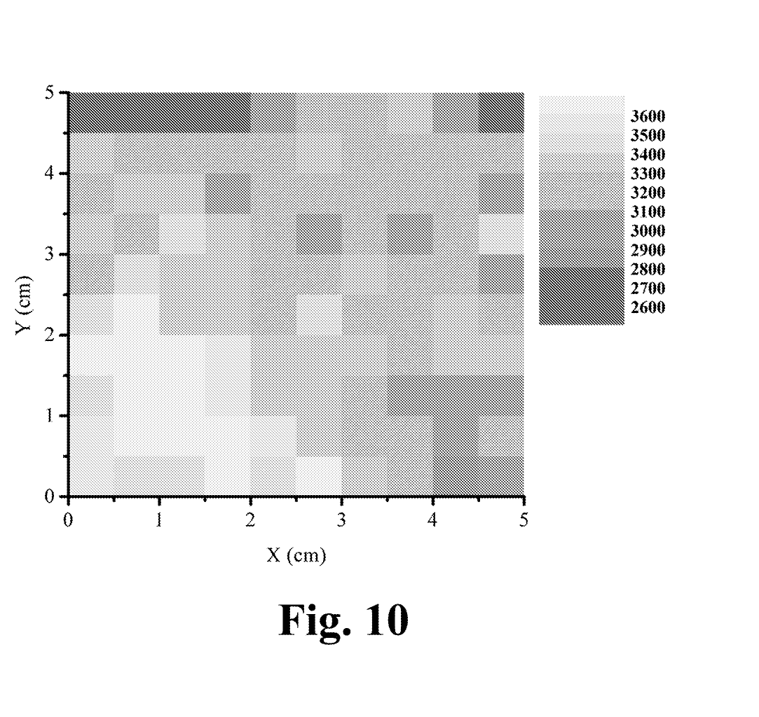

In practical application, uniformity of the SERS substrate contributes to the enhancement factor. An enhancement factor of 10⁶-10⁹ issufficiently high for single molecule detection. 1,2-BDT was used to investigate the uniformity of the SERS substrate 10 since 1,2-BDT forms a monolayer on gold surfaces. Under such circumstances, aggregation or poor distribution of the analyte could be precluded and the results would more likely reflect the intrinsic property of the substrate 10. The SERS substrate 10 in this example was triple processed gold nanoisland 15 arrays with 40 nm gold deposition. The dimension of this SERS substrate 10 was 5 cm by 5 cm. After the formation of a self-assembled 1,2 BDT monolayer, the 5 cm by 5 cm SERS substrate 10 was divided evenly into 100 grid cells (5 mm by 5 mm each). The intensity of peak at a band of 1030 cm⁻¹ from each grid cell was collected and plotted versus the substrate's 10 X-Y position, as shown in FIG. 10. The relative standard deviation (RSD) of the Raman intensity was estimated to be ˜7% over 100 grid cells.

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

What is claimed is:

1. A substrate for facilitating enhanced SERS analysis, comprising:
a first substrate surface; and
a plurality of discrete metal nanostructures disposed on the first substrate surface to define an array;
wherein each respective metal nanostructure is between about 10 nm and about 30 nm high and between about 15 nm and about 60 nm in diameter; and
wherein two adjacent respective metal nanostructures are separated by a gap of between about 20 nm to about 50 nm.

2. The substrate of claim 1 wherein the nanostructures are made from a material selected from the set including gold, silver, platinum, titanium, chromium, copper, nickel, and combinations thereof.

3. The device of claim 1 and further comprising a metallic coating partially filling in at least some of the gaps.

4. The device of claim 1 wherein the respective nanostructures are roughened to include secondary nanostructures extending therefrom.

5. The device of claim 4 wherein the secondary nanostructures are between about 5 nm and about 15 nm in diameter.

6. The device of claim 1 wherein the respective nanostructures are about 40 nm in diameter.

7. The device of claim 1 wherein the gaps are about 20 nanometers.

8. The device of claim 1 wherein the respective nanostructures are about 40 nm in diameter and wherein the gaps are about 20 nanometers.

9. The device of claim 1 wherein the nanostructures are gold.

10. The device of claim 9 wherein the gold nanostructures are substantially free of internal strain.

11. A surface enhanced Raman spectroscopy device, comprising:
a base substrate;
a plurality of gold nanostructures formed on the substrate; and
a plurality of gaps interspersed between the respective gold nanoparticles;
wherein the nanoparticles are about 40 nm across; and
wherein the gaps are about 20 nm across.

12. A method of producing a surface enhanced Raman spectroscopy device, comprising:
a) forming a plurality of gold nanostructures on a base substrate;
b) annealing the plurality of gold nanostructures;
c) increasing the size of the gold structures to define a plurality of second generation gold structures;
d) annealing the plurality of second generation gold nanostructures;
e) increasing the size of the second generation gold structures to define a plurality of third generation gold nanostructures; and
f) annealing the third generation gold nanostructures;
wherein the plurality of third generation gold nanostructures defines a plurality of gaps therebetween;
wherein each respective gap is between two or more adjacent gold nanostructures;
wherein each respective gold nanostructure is between about 30 nm and about 60 nm across;
wherein each respective gold nanostructure is between about 20 nm and about 30 nm in height.

13. The method of claim 12 wherein a), c), and e) are performed by sputtering a gold film of 5 nm nominal thickness onto a silicon substrate.

14. The device of claim 12 wherein b), d) and f) are performed by heating the substrate to 200 degrees Celsius for 2 hours in a forming gas atmosphere.

15. A method of producing a SERS device, comprising:
a) sputtering metal onto a base silicon substrate to define a first plurality of metal nanostructures; and
b) annealing the first plurality of metal nanostructures to define a plurality of first generation metal nanostructures;
wherein the plurality of first generation metal nanostructures are substantially evenly distributed;
wherein the plurality of first generation nanostructures define a plurality of substantially evenly distributed gaps therebetween;
wherein each respective first generation metal nanostructure is about 15 nm in diameter and about 10 nm high; and
wherein each respective gap contains between 0 and about 5 nm thick metal.

16. The method of claim 15 and further comprising:
c) sputtering metal onto a the plurality of first generation metal nanostructures to define a second plurality of metal nanostructures; and
d) annealing the second plurality of metal nanostructures to define a plurality of second generation metal nanostructures;
wherein each respective second generation metal nanostructure is about 25 nm in diameter and about 15 nm high; and
wherein each respective gap contains between 0 and about 10 nm thick metal.

17. The method of claim 16 and further comprising:
e) sputtering metal onto a the plurality of second generation metal nanostructures to define a third plurality of metal nanostructures; and
f) annealing the third plurality of metal nanostructures to define a plurality of third generation metal nanostructures;
wherein each respective third generation metal nanostructure is about 40 nm in diameter and about 25 nm high; and
wherein each respective gap contains between 0 and about 15 nm thick metal.

18. The method of claim 17 and further comprising:
g) sputtering a layer of metal between about 5 nm and about 10 nm thick onto a the plurality of third generation metal nanostructures to define a plurality of roughened nanostructures.

19. The method of claim 15 wherein the metal is selected from the group including gold, silver, platinum, copper, nickel, and combinations thereof.

20. The method of claim 17 wherein each respective third generation nanostructure includes layers of dissimilar metals.
